SEQUENCE LISTING (5 sequences)

SEQ ID NO: 1 - protein, 7 aa, synthetic construct
MEHFPGP

SEQ ID NO: 2 - protein, 7 aa, synthetic construct
MEHFRWG

SEQ ID NO: 3 - protein, 7 aa, synthetic construct
  MOD_RES 1: note = N-acetylmethionine
MEHFPGP

SEQ ID NO: 4 - protein, 7 aa, synthetic construct
  MOD_RES 1: note = N-acetylmethonine
  MOD_RES 7: note = Proline-amidate
MEHFPGP

SEQ ID NO: 5 - protein, 7 aa, synthetic construct
  MOD_RES 1: note = N-acetylmethionine
  MOD_RES 7: note = Proline-adamantane-CONH(2)
MEHFPGP

(19) United States
(12) Patent Application Publication
(10) Pub. No.: US 2025/0127843 A1
(43) Pub. Date: Apr. 24, 2025

(54) ADRENOCORTICOTROPIC HORMONE PEPTIDE COMPOSITIONS AND METHODS OF USE

(71) Applicant: FERRING B.V., Hoofddorp (NL)

(72) Inventors: Iuliia Vasilyevna Vlasenko, Moscow (RU); Mariya Andreyevna Merkulova, Moscow (RU)

(73) Assignee: FERRING B.V., Hoofddorp (NL)

(21) Appl. No.: 18/694,313

(22) PCT Filed: Jul. 26, 2022
(86) PCT No.: PCT/EP2022/070904

§ 371 Date: Mar. 21, 2024

(30) Foreign Application Priority Data
Sep. 22, 2021 (RU) 2021127814

(51) Int. Cl. A61K 38/08; A61K 47/02; A61K 47/10; A61K 47/12; A61K 47/18; A61K 47/26; A61K 47/38

(52) U.S. Cl. CPC A61K 38/08; A61K 47/02; A61K 47/10; A61K 47/12; A61K 47/183; A61K 47/186; A61K 47/26; A61K 47/38

(57) ABSTRACT

Described herein are aqueous pharmaceutical compositions comprising peptide Met-Glu-His-Phe-Pro-Gly-Pro (SEQ ID NO: 1), or a pharmaceutically acceptable derivative thereof, or a pharmaceutically acceptable salt of any thereof, and therapeutic methods using them.

### FIELD

Described herein are aqueous liquid pharmaceutical compositions comprising peptide Met-Glu-His-Phe-Pro-Gly-Pro (SEQ ID NO: 1) formulated for nasal administration, and therapeutic uses thereof.

### BACKGROUND

Met-Glu-His-Phe-Pro-Gly-Pro (SEQ ID NO: 1) is a modified synthetic analogue of adrenocorticotropic hormone ACTH₄₋₁₀ (Met-Glu-His-Phe-Arg-Trp-Gly; SEQ ID NO: 2), without hormonal activity. This modified synthetic analogue is known for its nootropic, neuroprotective, and neurogenic/neurorestorative properties and has been indicated in Russia for treating intellectual-mental disorders in vascular malformations of the brain, dyscirculatory encephalopathy, transient ischemic attack (TIA), neurotic disorders, glaucoma, optic nerve disorders, minimal brain dysfunctions in children, including attention deficit hyperactivity disorder (ADHD), and mental fatigue, for promoting recovery after craniocerebral injury, for promoting recovery after narcosis, and for stroke management or treatment after neurosurgery. In addition to these approved indications, the peptide has been proposed for use in treating multiple sclerosis, perinatal damage of CNS with atonic-astatic syndrome, and organic mental disorders in children.

An aqueous nasal formulation of Met-Glu-His-Phe-Pro-Gly-Pro (SEQ ID NO: 1) has been approved for use in Russia as SEMAX at two strengths, 0.1% and 1.0%. SEMAX is formulated with methyl paraben and purified water and provided as a colorless, transparent liquid in a 3 ml dropper bottle (a glass vial with a pipette), administered by instillation of nasal drops, which is an inconvenient delivery system that can lack dosing consistency and uniformity. SEMAX frequently cause nasal irritation, likely due to the hypotonic nature of the formulation and the presence of methyl paraben. SEMAX require storage at less than 10° C.

Thus, there remains a need for aqueous pharmaceutical compositions comprising Met-Glu-His-Phe-Pro-Gly-Pro (SEQ ID NO: 1), formulated for nasal administration.

### SUMMARY

Provided herein are aqueous pharmaceutical compositions comprising:
(a) peptide Met-Glu-His-Phe-Pro-Gly-Pro (SEQ ID NO: 1), or a pharmaceutically acceptable derivative thereof or a pharmaceutically acceptable salt of any thereof;
(b) an antioxidant;
(c) an osmolarity regulating agent;
(d) optionally, an antimicrobial preservative; and
(e) water,
wherein the pharmaceutical composition has a pH of from about 4.0 to about 7.0, and an osmolarity of from about 235 mOsm/L to about 380 mOsm/L.

In some embodiments, the pharmaceutical composition further comprises a buffering agent. The buffering agent may provide a buffer selected from one or more of an acetate buffer, a citrate buffer, a phosphate buffer, a phosphate-citrate buffer, and a succinate buffer. In some embodiments, the buffering agent provides an acetate buffer.

In some embodiments, the pharmaceutical composition has a pH of from about 4.5 to about 5.0. In some embodiments, the pharmaceutical composition has a pH of from about 4.0 to about 6.5. In some embodiments, the pharmaceutical composition has a pH of from about 4.0 to about 5.5. In some embodiments, the pharmaceutical composition has a pH of about 4.5.

In some embodiments, the peptide, pharmaceutically acceptable derivative thereof, or pharmaceutically acceptable salt of any thereof, is present at a concentration of from about 0.1 mg/ml to about 100 mg/ml, based on the free peptide. In some embodiments, the peptide, pharmaceutically acceptable derivative thereof, or pharmaceutically acceptable salt of any thereof, is present at a concentration of from about 0.5 mg/ml to about 50 mg/ml, based on the free peptide. In some embodiments, the peptide, pharmaceutically acceptable derivative thereof, or pharmaceutically acceptable salt of any thereof, is present at a concentration of from about 0.8 mg/ml to about 12 mg/ml, based on the free peptide.

In some embodiments, the antioxidant is one or more selected from an amino acid, a vitamin, a vitamin derivative, an inorganic salt, and a phenol. In some embodiments, the antioxidant comprises an amino acid selected from methionine, cysteine, histidine, asparagine, glycine, alanine, valine, phenylalanine, and cystine, optionally wherein the amino acid is the L-form. In some embodiments, the antioxidant comprises methionine, optionally wherein the methionine is the L-form. In some embodiments, the antioxidant comprises ascorbic acid or a derivative thereof. In some embodiments, the antioxidant comprises an inorganic salt selected from sodium thiosulfate, sodium metabisulfite, and potassium metabisulfite. In some embodiments, the antioxidant comprises a phenol selected from thymol and hydroxyanisole.

In some embodiments, the antioxidant is present at a concentration ratio of antioxidant to peptide or pharmaceutically acceptable derivative thereof or pharmaceutically acceptable salt of any thereof, of from about 0.1:1 to about 10:1, based on the total amount of antioxidant present and the amount of free peptide. In some embodiments, the antioxidant is present at a concentration ratio of antioxidant to peptide or pharmaceutically acceptable derivative thereof or pharmaceutically acceptable salt of any thereof, of from about 0.1:1 to about 5:1, based on the total amount of antioxidant present and the amount of free peptide. In some embodiments, the antioxidant is present at a concentration ratio of antioxidant to peptide or pharmaceutically acceptable derivative thereof or pharmaceutically acceptable salt of any thereof, of about 1:1, based on the total amount of antioxidant present and the amount of free peptide.

In some embodiments, the antimicrobial preservative is present and is one or more selected from a quaternary ammonium compound, sorbic acid, glycerol, benzyl alcohol, chlorobutanol, phenyl ethanol, and benzoic acid or a salt thereof. In some embodiments, the antimicrobial preservative comprises the quaternary ammonium compound benzalkonium chloride. In some embodiments, the antimicrobial preservative is present at a concentration of from about 0.01 mg/ml to about 2 mg/ml. In some embodiments, the antimicrobial preservative is present at a concentration of from about 0.02 mg/ml to about 1.2 mg/ml.

In some embodiments, the osmolarity regulating agent is one or more selected from an inorganic salt and a polyol. In some embodiments, the osmolarity regulating agent comprises one or more selected from sorbitol, glycerol, mannitol, maltitol, dextrose, and sucrose. In some embodiments, the osmolarity regulating agent comprises an inorganic salt selected from sodium chloride and sodium sulfate. In some embodiments, the osmolarity regulating agent comprises sodium chloride and sorbitol. In some embodiments, the osmolarity regulating agent comprises glycerol. In some embodiments, the osmolarity regulating agent is present at a concentration of from about 0.01 mg/ml to about 100 mg/ml.

In some embodiments, the pharmaceutical composition further comprises a chelating agent. In some embodiments, the chelating agent is one or more selected from a tetrabasic carboxylic acid, a tribasic carboxylic acid, a dibasic carboxylic acid, a dibasic hydroxycarboxylic acid, and pharmaceutically acceptable salts thereof. In some embodiments, the chelating agent comprises edetate disodium (EDTA). In some embodiments, the chelating agent is present at a concentration of from about 0.01 mg/ml to about 5 mg/ml. In some embodiments, the chelating agent is present at a concentration of from about 0.3 mg/ml to about 2 mg/ml.

In some embodiments, the pharmaceutical composition further comprises a thickening agent. In some embodiments, the thickening agent is one or more selected from cellulose and cellulose derivatives (optionally hydroxyethyl cellulose, hydroxypropyl methylcellulose, microcrystalline cellulose, and/or carboxymethylcellulose), polyvinylpyrrolidone, acrylic acid polymers and copolymers, polyoxyethylene-polyoxypropylene block copolymers, and salts of any thereof. In some embodiments, the thickening agent is present at a concentration of from about of about 0.1 mg/ml to about 100 mg/ml. In some embodiments, the thickening agent is present at a concentration of from about 0.5 mg/ml to about 5 mg/ml. In some embodiments, the composition has a viscosity of about 5 mPa·s or less.

In some embodiments, the antioxidant comprises L-methionine; the osmolarity regulating agent comprises one or both of sorbitol and sodium chloride; the antimicrobial preservative is present and comprises benzalkonium chloride; if present, the thickening agent comprises one or both of hydroxypropyl methylcellulose and hydroxyethyl cellulose; if present, the chelating agent comprises edetate disodium; and, if present, the buffering agent comprises an acetate buffer. In some embodiments, the composition comprises about 1 mg/ml or about 10 mg/ml of the peptide, or pharmaceutically acceptable derivative thereof, or pharmaceutically acceptable salt of any thereof, based on the amount of free peptide. In some embodiments comprising methionine as the antioxidant, the ratio of the concentration of methionine to the concentration of the peptide or pharmaceutically acceptable derivative thereof or pharmaceutically acceptable salt of any thereof, is about 1:1, based on the amount of free peptide.

In accordance with any of the foregoing embodiments, the composition may comprise an acetate salt of the peptide. Additionally or alternatively, in accordance with any of the foregoing embodiments, the composition may comprise a pharmaceutically acceptable derivative of the peptide having an N-terminal acetyl moiety, a C-terminal amidate moiety or a C-terminal adamantane moiety, or both an N-terminal acetyl moiety and a C-terminal amidate moiety or a C-terminal adamantane moiety. In some embodiments, the composition comprises a pharmaceutically acceptable derivative of the peptide selected from:
N-acetyl-Met-Glu-His-Phe-Pro-Gly-Pro;
N-acetyl-Met-Glu-His-Phe-Pro-Gly-Pro-amidate, and
N-acetyl-Met-Glu-His-Phe-Pro-Gly-Pro-adamantane-CONH₂.

In accordance with any embodiments, the composition may not include any parabens or salts thereof (e.g., may be free of parabens and salts thereof).

In accordance with any embodiments, the composition may be provided in or packaged with a nasal spray drug delivery device. In accordance with any embodiments, the nasal spray drug delivery device may be configured to deliver a target dose volume of about 100 μL.

In another aspect, provided herein are methods of treatment, comprising administering any pharmaceutical composition described herein to a subject in need thereof by intranasal administration. In some embodiments, the pharmaceutical composition is administered from a nasal spray drug delivery device. In some embodiments, the pharmaceutical composition is administered at a target dose volume of about 100 μL. In some embodiments, the method is for treating one or more conditions selected from intellectual-mental disorders in vascular malformations of the brain, dyscirculatory encephalopathy, transient ischemic attack (TIA), neurotic disorders, glaucoma, optic nerve disorders, minimal brain dysfunctions in children, attention deficit hyperactivity disorder (ADHD), mental fatigue, multiple sclerosis, perinatal damage of CNS with atonic-astatic syndrome, and organic mental disorders in children, or for promoting recovery after craniocerebral injury or for promoting recovery after narcosis, or for stroke management or treatment after neurosurgery.

In another aspect, provided herein are aqueous pharmaceutical compositions as described herein, optionally provided in or packaged with a nasal spray delivery device as described herein, for use in treating one or more conditions selected from intellectual-mental disorders in vascular malformations of the brain, dyscirculatory encephalopathy, transient ischemic attack (TIA), neurotic disorders, glaucoma, optic nerve disorders, minimal brain dysfunctions in children, attention deficit hyperactivity disorder (ADHD), mental fatigue, multiple sclerosis, perinatal damage of CNS with atonic-astatic syndrome, and organic mental disorders in children, or for promoting recovery after craniocerebral injury or for promoting recovery after narcosis, or for stroke management or treatment after neurosurgery.

In another aspect, provided herein are uses of peptide Met-Glu-His-Phe-Pro-Gly-Pro (SEQ ID NO: 1), or a pharmaceutically acceptable derivative thereof, or a pharmaceutically acceptable salt of any thereof, in the preparation of a medicament for treating one or more conditions selected from intellectual-mental disorders in vascular malformations of the brain, dyscirculatory encephalopathy, transient ischemic attack (TIA), neurotic disorders, glaucoma, optic nerve disorders, minimal brain dysfunctions in children, attention deficit hyperactivity disorder (ADHD), mental fatigue, multiple sclerosis, perinatal damage of CNS with atonic-astatic syndrome, and organic mental disorders in children, or for promoting recovery after craniocerebral injury or for promoting recovery after narcosis, or for stroke management or treatment after neurosurgery, wherein the medicament comprises any one of the aqueous pharmaceutical composition described herein, optionally provided in or packaged with a nasal spray delivery device as described herein.

### DETAILED DESCRIPTION

The present disclosure provides aqueous pharmaceutical compositions of Met-Glu-His-Phe-Pro-Gly-Pro (SEQ ID NO: 1) (also referred to herein as “the peptide”) formulated for nasal administration that are isotonic and formulated without parabens. Thus, the disclosed compositions are non-irritating, or at least less irritating than SEMAX. Additionally, in some embodiments, the disclosed compositions exhibit room temperature stability. Additionally, in some embodiments, the disclosed compositions are provided in or packaged with a nasal drug delivery device that provides consistent and uniform dose delivery. As described in more detail below, the compositions generally include (a) peptide Met-Glu-His-Phe-Pro-Gly-Pro (SEQ ID NO: 1), or a pharmaceutically acceptable derivative thereof, or a pharmaceutically acceptable salt of any thereof; (b) an antioxidant; (c) an osmolarity regulating agent; (d) optionally, an antimicrobial preservative; and (e) water. The compositions have a pH of from about 4.0 to about 7.0 and an osmolarity of from about 235 mOsm/L to about 380 mOsm/L.

### Definitions

Technical and scientific terms used herein have the meanings commonly understood by one of ordinary skill in the art to which the present invention pertains, unless otherwise defined. Any suitable materials and/or methods known to those of ordinary skill in the art can be utilized in carrying out the present invention in view of the guidance provided herein; however, specific materials and methods are described for illustrative purposes. Materials, reagents and the like to which reference is made in the following description and examples are obtainable from commercial sources, unless otherwise noted.

As used herein, the singular forms “a,” “an,” and “the” designate both the singular and the plural, unless expressly stated to designate the singular only.

As used herein, “about” when used with a numerical value means the numerical value stated as well as plus or minus 10% of the numerical value. For example, “about 10” should be understood as both “10” and “9-11.”

As used herein, a phrase in the form “A/B” or in the form “A and/or B” means (A), (B), or (A and B); a phrase in the form “at least one of A, B, and C” means (A), (B), (C), (A and B), (A and C), (B and C), or (A, B, and C).

As used herein, the terms “comprising,” “including,” and “containing” are used expansively to mean that the described compositions, methods, or kits include at least the stated elements, and may include other elements that are not specified. The phrase “consisting essentially of” is used to include those elements specifically recited and additional elements that do not materially affect the basic and novel characteristics of the claimed invention, such as ingredients that do not materially undermine the stability of the peptide in the composition or the suitability of the composition for nasal administration.

As used herein, “subject” denotes any mammal, including humans. For example, a subject may be suffering from or at risk of developing a condition that can be treated or prevented with the peptide, or may be administered the peptide for other purposes.

The terms “administer,” “administration,” and “administering” as used herein refer to providing, giving, dosing and/or prescribing, such as by a health professional or his or her authorized agent or under his or her direction, and putting into, taking, or consuming, such as by a health professional or the subject.

The terms “treat,” “treating,” and “treatment” as used herein include alleviating, abating or ameliorating a disease or condition or one or more symptoms thereof, whether or not the disease or condition is considered to be “cured” or “healed,” and whether or not all symptoms are resolved.

As used herein, the phrases “therapeutically effective amount” and “therapeutically effective dose” refer to an amount or dose that provides the specific pharmacological effect for which the composition is administered in a subject in need of such treatment. It is emphasized that a therapeutically effective amount will not always be effective in treating the targeted condition, even though such amount or dose is deemed to be a therapeutically effective amount or dose by those of skill in the art. For convenience only, exemplary doses and therapeutically effective amounts are provided below with reference to adult human subjects. Those skilled in the art can adjust such amounts in accordance with standard practices as needed to treat a specific subject and/or condition/disease.

### The Peptide Active Ingredient

The active ingredient of the compositions described herein is the peptide Met-Glu-His-Phe-Pro-Gly-Pro (SEQ ID NO: 1), or a pharmaceutically acceptable derivative thereof, or a pharmaceutically acceptable salt of any thereof (Sometimes referred to herein collectively as the “active ingredient”) The molecular formula of the peptide is C₃₇H₅₁N₉O₁₀S, and the molecular weight of the peptide is 813.92 (free base). It is registered under CAS Registry Number 80714-61-0. The structural formula of the peptide is set forth below:

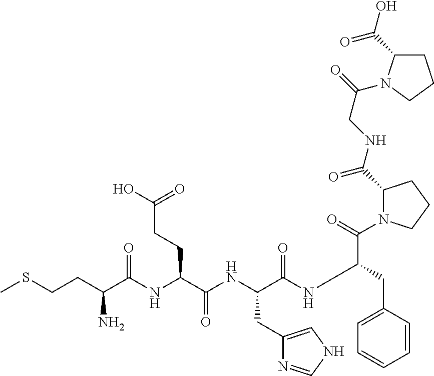 [SMILES: CSCC[C@H](N)C(=O)N[C@@H](CCC(=O)O)C(=O)N[C@@H](Cc1c[nH]cn1)C(=O)N[C@@H](Cc1ccccc1)C(=O)N1CCC[C@H]1C(=O)NCC(=O)N1CCC[C@H]1C(=O)O]

For embodiments using a pharmaceutically acceptable salt of the peptide, any pharmaceutically acceptable salt suitable for nasal administration can be used. Examples of pharmaceutically acceptable salts include acid salts such as hydrochlorides and basic salts such as alkali metal salts, alkaline earth metal salts, and ammonium salts. More specific examples include acid addition salts, e.g., salts formed by reaction with hydrohalogen acids such as hydrochloric acid or mineral acids, such as sulphuric acid, phosphoric acid and nitric acid, as well as aliphatic, alicyclic, aromatic or heterocyclic sulphonic or carboxylic acids, such as formic acid, acetic acid, propionic acid, succinic acid, glycolic acid, lactic acid, malic acid, tartaric acid, citric acid, benzoic acid, ascorbic acid, maleic acid, hydroxymaleic acid, pyruvic acid, p-hydroxybenzoic acid, embonic acid, methanesulphonic acid, ethanesulphonic acid, hydroxyethanesulphonic acid, halobenzenesulphonic acid, trifluoroacetic acid, trifluoromethanesulphonic acid, toluenesulphonic acid and naphthalenesulphonic acid. The pharmaceutically acceptable salt may be an acetate salt.

For embodiments using a pharmaceutically acceptable derivative of the peptide, any pharmaceutically acceptable derivative suitable for nasal administration can be used. Pharmaceutically acceptable derivatives include derivatives of the molecule modified with one or more moieties to achieve or improve a desired property, such as one or more of increased half-life, increased blood-brain-barrier permeability, and increased resistance to degradation in vivo or during storage. For example, the pharmaceutically acceptable derivative may comprise one or more of an acetyl moiety, an amidate moiety, and an adamantane moiety. For example, the pharmaceutically acceptable derivative may comprise an N-terminal acetyl moiety. Additionally or alternatively, the pharmaceutically acceptable derivative may comprise a C-terminal amidate moiety or a C-terminal adamantane moiety. For example, the pharmaceutically acceptable derivative may be N-acetyl-[Peptide], N-acetyl-[Peptide]-amidate, or N-acetyl-[Peptide]-adamantane-CONH₂.

The peptide is reported to have neuroprotective effects, and has been shown to bind to the nuclear membranes of nerve cells of the basal forebrain, hippocampus and cerebellum, and to specifically bind to the membranes of the basal ganglia and cerebral cortex. The peptide also has been shown to reduce the number of damaged cells under conditions of oxidative stress caused by short-term incubation with hydrogen peroxide, significantly increase the survival of cerebellar granule neurons during glutamate neurotoxicity, and increase levels of Bcl-2, which has an important role in survival-signal pathways that inhibit apoptosis.

Additionally, the peptide has been reported to have a neurotrophic effect, and has been shown to have an effect on the expression of neurotrophins (likely through action on mGlu-receptors), resulting in rapid induction of the transcription of the neurotrophin BDNF and NGF genes, significant increase in the expression of the nuclear protein PCNA (which participates in the preparation for cell division), and promotion of the differentiation and maintenance of the viability and function of peripheral and central neurons.

The compositions described herein may comprise the active ingredient (the peptide or pharmaceutically acceptable derivative thereof or pharmaceutically acceptable salt of any thereof) in any suitable amount. For example, a composition as described herein may comprise the active ingredient at a concentration of from about 0.1 mg/ml to about 100 mg/ml, including about 0.1 mg/ml, about 1 mg/ml, about 10 mg/ml, about 100 mg/ml, and any value therebetween, all based on the amount of the free peptide. A composition as described herein may comprise from about 0.5 mg/ml to about 50 mg/ml of the active ingredient, including about 0.5 mg/ml, about 25 mg/ml, and about 50 mg/ml, and any value therebetween, all based on the amount of the free peptide. A composition as described herein may comprise from about 0.8 mg/ml to about 12 mg/ml of the active ingredient, including about 0.8 mg/ml, about 10 mg/ml, and about 12 mg/ml, and any value therebetween, all based on the amount of the free peptide. A composition as described herein may comprise about 10 mg/ml of the active ingredient, based on the amount of the free peptide. Those skilled in the can determine corresponding concentrations of peptide derivatives or pharmaceutically acceptable salts based on the relative molecular weights of the peptide and peptide derivative or peptide salt.

The compositions described herein may be formulated to provide a therapeutically effective dose of the active ingredient (peptide or pharmaceutically acceptable derivative thereof or pharmaceutically acceptable salt of any thereof) in a volume suitable for nasal administration. The therapeutically effective dose of the active ingredient (etc.) may vary with the condition and/or subject being treated. The therapeutically effective dose of the active ingredient may be from about 10 μg/day to about 25,000 μg/day, from about 100 μg/day to about 20,000 μg/day, from about 200 μg/day to about 20,000 μg/day, from about 6,000 μg/day to about 12,000 μg/day, including about 100 μg/day, about 200 μg/day, about 6,000 μg/day, about 12,000 μg/day, about 20,000 μg/day, and any value therebetween, all based on the amount of free peptide. Those skilled in the can determine corresponding doses of peptide derivatives or pharmaceutically acceptable salts. For example, a corresponding dose of a pharmaceutically acceptable salt of the peptide can be calculated based on the relative molecular weights of the peptide and peptide salt. A corresponding dose of a derivative of the peptide can be calculated based on the relative molecular weights of the peptide and peptide derivative, or, if appropriate, can be adjusted if the peptide derivative has a greater bioavailability or longer half-life than the peptide.

As noted above, the compositions described herein typically include (a) the peptide, or a pharmaceutically acceptable derivative thereof, or a pharmaceutically acceptable of any salt thereof; (b) an antioxidant; (c) an osmolarity regulating agent; (d) optionally, an antimicrobial preservative; and (e) water, a pH of from about 4.0 to about 7.0, and an osmolarity of from about 235 mOsm/L to about 380 mOsm/L. The peptide (etc.) having been discussed above, the other components are discussed in turn below.

In advantageous embodiments, a composition as described herein is not formulated with and does not include any parabens or salts thereof, such as any one or more of methyl paraben, propyl paraben, butyl paraben, ethyl paraben, and salts of any one thereof. Thus, in some embodiments, a composition as described herein does not include methyl paraben and does not include any salt thereof (e.g., does not include methyl paraben sodium). In some embodiments, a composition as described herein does not include any of methyl paraben, propyl paraben, butyl paraben, ethyl paraben, and does not include any salts of any thereof. By formulating the composition without parabens, the composition may be less irritating.

### Antioxidants

The compositions described herein comprise an antioxidant. The antioxidant reduces or prevents oxidation of the peptide (or the pharmaceutically acceptable derivative thereof or the pharmaceutically acceptable salt of any thereof). The antioxidant may be any suitable (e.g., suitable for nasal administration and compatible with other components of the composition) and effective antioxidant, and may be one or more selected from an amino acid, a vitamin, a vitamin derivative, an inorganic salt, and a phenol. Examples of suitable amino acid antioxidants include, but are not limited to, methionine, cysteine, histidine, asparagine, glycine, alanine, valine, phenylalanine, and cystine. In some embodiments, the amino acid is the L-form. Examples of suitable vitamin or vitamin derivative antioxidants include, but are not limited to, ascorbic acid and derivatives thereof. Examples of suitable inorganic salt antioxidants include, but are not limited to, sodium thiosulfate, sodium metabisulfite, and potassium metabisulfite. Examples of suitable phenol antioxidants, include but are not limited to, thymol and hydroxyanisole. In some embodiments, the antioxidant comprises methionine, optionally wherein the methionine is L-methionine.

The compositions described herein may comprise the antioxidant at any suitable amount, such as any amount effective to reduce or prevent oxidation of the peptide, or the pharmaceutically acceptable derivative thereof or the pharmaceutically acceptable salt of any thereof. In some embodiments, the antioxidant is present at a concentration ratio of antioxidant to the active ingredient (the peptide or pharmaceutically acceptable derivative thereof or pharmaceutically acceptable salt of any thereof) of from about 0.1:1 to about 10:1, based on the total amount of antioxidant(s) present, including about 0.1:1, about 1:1, about 5:1, about 10:1, and any value therebetween, all based on the amount of the free peptide. In some embodiments, the antioxidant is present at a concentration ratio of antioxidant to active ingredient of from about 0.1:1 to about 5:1, based on the total amount of antioxidant(s) present and the amount of the free peptide. In some embodiments, the antioxidant is present at a concentration ratio of antioxidant to active ingredient of about 1:1, based on the total amount of antioxidant(s) present and the amount of the free peptide.

As noted above, in some embodiments, the antioxidant is or comprises methionine (e.g., L-methionine). The methionine may be present at a concentration ratio of methionine to active ingredient of from about 0.1:1 to about 10:1, based on the total amount of methionine present, including about 0.1:1, about 1:1, about 5:1, about 10:1, and any value therebetween, based on the amount of free peptide. In some embodiments, methionine is present at a concentration ratio of methionine to active ingredient of from about 0.1:1 to about 5:1, based on the total amount of methionine present and the amount of the free peptide. In some embodiments, methionine is present at a concentration ratio of methionine to peptide, or the pharmaceutically acceptable derivative thereof, or the pharmaceutically acceptable salt of any thereof, of about 1:1, based on the amount of free peptide present.

### Osmolarity Regulating Agent

The compositions described herein comprise an osmolarity regulating agent. The osmolarity regulating agent is used to ensure isotonity of the composition, thereby reducing the risk of nasal irritation. The osmolarity regulating agent may be any suitable (e.g., suitable for nasal administration and compatible with other components of the composition) and effective osmolarity regulating agent, and may be one or more selected from an inorganic salt and a polyol. Examples of suitable polyol osmolarity regulating agents include, but are not limited to, sorbitol, glycerol, mannitol, maltitol, dextrose, and sucrose. (These polyols also may stabilize the peptide in the compositions described herein.) Examples of suitable inorganic salt osmolarity regulating agents include, but are not limited to, sodium chloride and sodium sulfate. In some embodiments, the osmolarity regulating agent is or comprises sodium chloride. In some embodiments, the osmolarity regulating agent is or comprises sorbitol. In some embodiments, the osmolarity regulating agent is or comprises sodium chloride and sorbitol. In some embodiments, the osmolarity regulating agent is or comprises glycerol.

The compositions described herein may comprise the osmolarity regulating agent at any suitable amount, such as any amount effective to maintain isotonicity, such as to provide or maintain a target osmolarity. The compositions described herein may comprise the osmolarity regulating agent at a concentration of from about 0.01 mg/ml to about 100 mg/ml, or from about 0.01 mg/ml to about 10 mg/ml, including about 0.1 mg/ml, about 10 mg/ml, about 100 mg/ml, and any value therebetween. In some embodiments, the osmolarity regulating agent is present at a concentration of from about 0.01 mg/ml to about 100 mg/ml. In some embodiments, the osmolarity regulating agent is present at a concentration of from about 0.01 mg/ml to about 10 mg/ml.

As noted above, the compositions described herein may comprise the osmolarity regulating agent at an amount effective to provide or maintain a target osmolarity, such as an osmolarity of from about 235 mOsm/L to about 380 mOsm/L, including about 235 mOsm/L, about 239 mOsm/L, about 376 mOsm/L, about 380 mOsm/L, and any value therebetween. In some embodiments, the osmolarity regulating agent is present at a concentration effective to provide or maintain an osmolarity of from about 235 mOsm/L to about 376 mOsm/L.

### Antimicrobial Preservatives

The compositions described herein optionally may comprise an antimicrobial preservative. The antimicrobial preservative may be any suitable agent (e.g., suitable for nasal administration and compatible with other components of the composition) effective to reduce or prevent microbial contamination in the composition. As noted above, however, in advantageous embodiments the antimicrobial preservative does not comprises a paraben or a salt thereof, such as methyl paraben, propyl paraben, butyl paraben, ethyl paraben, and the salt of any thereof. The antimicrobial preservative may be one or more selected from a quaternary ammonium compound, sorbic acid, glycerol, benzyl alcohol, chlorobutanol, phenyl ethanol, and benzoic acid or a salt thereof. The antimicrobial preservative may comprise a quaternary ammonium compound. In some embodiments, the antimicrobial preservative comprises the quaternary ammonium compound benzalkonium chloride. Without wanting to be bound by theory, it is believed the antimicrobial preservative also may slow down ciliary beat frequency, thereby decreasing the speed of nasal mucosa clearance and increasing retention time of the compositions described herein in the nasal cavity.

The compositions described herein may comprise the antimicrobial preservative at any suitable amount, such as any amount effective to reduce or prevent microbial contamination in the composition. The antimicrobial preservative may be present at a concentration from about 0.01 mg/ml to about 2 mg/ml, including about 0.01 mg/ml, about 0.02 mg/ml, about 0.05 mg/ml, about 0.2 mg/ml, about 1.2 mg/ml, about 2 mg/ml, and any value therebetween. In some embodiments, the antimicrobial preservative is present at a concentration of from about 0.02 mg/ml to about 1.2 mg/ml. In some embodiments, the antimicrobial preservative is present at a concentration of from about 0.02 mg/ml to about 0.2 mg/ml.

In some embodiments, the antimicrobial preservative is or comprises benzalkonium chloride. Benzalkonium chloride may be present at a concentration from about 0.01 mg/ml to about 2 mg/ml, including about 0.01 mg/ml, about 0.02 mg/ml, about 0.05 mg/ml, about 0.2 mg/ml. about 1.2 mg/ml, about 2 mg/ml, and any value therebetween. In some embodiments, the benzalkonium chloride is present at a concentration of from about 0.02 mg/ml to about 1.2 mg/ml. In some embodiments, the benzalkonium chloride is present at a concentration of from about 0.02 mg/ml to about 0.2 mg/ml.

### Buffering Agents

The compositions described herein optionally may comprise a buffering agent. The buffering agent may stabilize the pH of the compositions at a target pH, such as a pH at which the peptide is stable, and additionally or alternatively, a pH at which the composition as a whole is stable. As noted above, the compositions described herein have a pH of from 4.0 to about 7.0, including a pH from about 4.5 to about 5.0, from about 4.0 to about 6.5, and from about 4.0 to about 5.5. Thus, the buffering agent may be any suitable buffering agent (e.g., suitable for nasal administration and compatible with other components of the composition) effective to maintain such a pH, including a pH of about 4.0, about 4.5, about 5.0, about 5.5, about 6.0, about 6.5, about 7.0, and any value therebetween. In some embodiments, the buffering agent provides a composition having a pH of about 4.5.

The buffering agent may be a salt, acid, base, or other compound that, when formulated in the composition provides an acetate buffer, a citrate buffer, a phosphate buffer, a phosphate-citrate buffer, or a succinate buffer. In some embodiments, the buffering agent provides an acetate buffer, such as one or more of acetic acid and sodium acetate. In some embodiments, the buffering agent provides a citrate buffer, such as one or more of citric acid and sodium citrate. In some embodiments, the buffering agent provides a phosphate buffer, such as one or more of disodium hydrogen phosphate and potassium dihydrogen phosphate. In some embodiments, the buffering agent provides a phosphate-citrate buffer, such as citric acid and disodium hydrogen phosphate. In some embodiments, the buffering agent provides a succinate buffer, such as succinic acid and sodium hydroxide. Those skilled in the art will appreciate that buffering agents can be used in hydrate form, such as citric acid monohydrate, tri-sodium citrate dihydrate, disodium hydrogen phosphate 12-hydrate, sodium acetate trihydrate, etc.

When a composition as described herein does not include a buffering agent, the composition still may have a pH of from 4.0 to about 7.0, including from about 4.5 to about 5.0. In some embodiments, a composition that does not include a buffering agent has a pH of about 4.5 or about 5.0.

### Chelating Agents

The compositions described herein optionally may comprise a chelating agent. The chelating agent may stabilize the formulation by reducing or preventing metal-induced oxidation of the peptide or other components. The chelating agent may be any suitable (e.g., suitable for nasal administration and compatible with other components of the composition) chelating agent. The chelating agent may be one or more selected from a tetrabasic carboxylic acid, a tribasic carboxylic acid, a dibasic carboxylic acid, a dibasic hydroxycarboxylic acid, and pharmaceutically acceptable salts of any thereof. In some embodiments, the chelating agent comprises edetate disodium (EDTA).

The compositions described herein may comprise the chelating agent in any suitable amount, such as any amount effective to reduce or prevent metal-induced oxidation of the peptide or other components of the composition. The chelating agent may be present at a concentration of from about 0.01 mg/ml to about 5 mg/ml, including about 0.01 mg/ml, about 0.1 mg/ml, about 0.3 mg/ml, about 2 mg/ml, about 5 mg/ml, and any value therebetween. In some embodiments, the chelating agent is present at a concentration of from about 0.1 mg/ml to about 5 mg/ml. In some embodiments, the chelating agent is present at a concentration of from about 0.3 mg/ml to about 2 mg/ml. In some embodiments, the chelating agent is present at a concentration of about 0.5 mg/ml.

In some embodiments, the chelating agent is present and is or comprises EDTA. EDTA may be present at a concentration of from about 0.01 mg/ml to about 5 mg/ml, including about 0.01 mg/ml, about 0.1 mg/ml, about 0.3 mg/ml, about 2 mg/ml, about 5 mg/ml, and any value therebetween. In some embodiments, EDTA is present at a concentration of from about 0.1 mg/ml to about 5 mg/ml. In some embodiments, EDTA is present at a concentration of from about 0.3 mg/ml to about 2 mg/ml. In some embodiments, EDTA is present at a concentration of about 0.5 mg/ml.

### Thickening Agents

The compositions described herein optionally may comprise a thickening agent. A thickening agent may be used to increase the viscosity of the composition, or, additionally or alternatively, provide mucoadhesive properties that increase the retention time of the composition in the nasal cavity. Furthermore, when the composition is used with a nasal spray device (as described in more detail below), the thickening agent may help create a more narrow spray pattern to target the upper (including olfactory) region of the nasal cavity for nose-to-brain delivery. The thickening agent may be any suitable (e.g., suitable for nasal administration and compatible with other components of the composition) thickening agent. The thickening agent may be one or more selected from cellulose and cellulose derivatives (such as hydroxyethyl cellulose, hydroxypropyl methylcellulose, microcrystalline cellulose, and carboxymethylcellulose), polyvinylpyrrolidone, acrylic acid polymers and copolymers, polyoxyethylene-polyoxypropylene block copolymers, and pharmaceutically acceptable salts of any thereof. In some embodiments, the thickening agent is or comprises hydroxyethyl cellulose. In some embodiments, the thickening agent is or comprises hydroxypropyl methylcellulose.

The compositions described herein may comprise the thickening agent at any suitable amount, such as any amount effective to achieve a target viscosity or, additionally or alternatively, any amount effective to achieve target mucoadhesive properties, or, additionally or alternatively, any amount effective to achieve a target nasal spray pattern. The thickening agent may be present at a concentration of from about 0.1 mg/ml to about 100 mg/ml, including about 0.1 mg/ml, about 0.5 mg/ml, about 5 mg/ml, about 10 mg/ml, about 100 mg/ml, and any value therebetween. In some embodiments, the thickening agent is present at a concentration of from about 0.1 mg/ml to about 10 mg/ml. In some embodiments, the thickening agent is present at a concentration of from about of about 0.5 mg/ml to about 5 mg/ml.

The compositions described herein may comprise the thickening agent at an amount that provides or results in a composition having a viscosity of about 20 mPa·s or less, including a viscosity of about 20 mPa·s, about 15 mPa·s, about 10 mPa·s, about 5 mPa·s, about 1.5 mPa·s, or less, or any value therebetween. In some embodiments, the composition (with or without thickening agent) has a viscosity of about 5 mPa·s or less. In some embodiments, the composition (with or without thickening agent) has a viscosity of about 1.5 mPa·s or less. In some embodiments, the composition (with or without thickening agent) has a viscosity of from about 0.5 mPa·s to about 5 mPa·s. In some embodiments, the composition (with or without thickening agent) has a viscosity of about 1.5 mPa·s. In some embodiments, a composition as described herein prepared without a thickening agent has a viscosity of from about 1.00 mPa·s to about 1.25 mPa·s.

### Exemplary Compositions

The following are disclosed as specific illustrative, non-limiting, embodiments of compositions as described herein.

Provided in one aspect are aqueous pharmaceutical compositions of peptide Met-Glu-His-Phe-Pro-Gly-Pro (SEQ ID NO: 1), or a pharmaceutically acceptable derivative thereof or a pharmaceutically acceptable salt of any thereof, comprising:
the peptide, or pharmaceutically acceptable derivative thereof or pharmaceutically acceptable salt of any thereof, optionally in an amount of about 1 mg/ml or about 10 mg/ml, based on the amount of free peptide;
L-methionine as an antioxidant, wherein the ratio of the concentration of L-methionine to the concentration of the peptide, or pharmaceutically acceptable derivative thereof or pharmaceutically acceptable salt of any thereof, is about 1:1, based on the amount of free peptide;
one or both sorbitol and sodium chloride as osmolarity regulating agents;
optionally, benzalkonium chloride as an antimicrobial preservative;
optionally, one or both of hydroxypropyl methylcellulose and hydroxyethyl cellulose as thickening agents; and
optionally a buffering agent that provides an acetate buffer,
   wherein the composition has a pH from about 4.0 to about 7.0 and an osmolarity of from about 235 mOsm/L to about 380 mOsm/L.

In specific embodiments, the aqueous pharmaceutical composition comprises:
about 1 mg/ml or about 10 mg/ml of the peptide, or pharmaceutically acceptable derivative thereof or pharmaceutically acceptable of any salt thereof, based on the amount of free peptide;
about 1 mg/ml or about 10 mg/ml of L-methionine, wherein the ratio of the concentration of methionine to the concentration of the peptide, or pharmaceutically acceptable derivative thereof or pharmaceutically acceptable salt of any thereof, is about 1:1, based on the amount of free peptide;
sodium chloride as an osmolarity regulating agent;
about 0.2 mg/ml of benzalkonium chloride;
hydroxyethyl cellulose as a thickening agent, optionally at an amount that provides a viscosity of about 5 mPa·s or less; and
an acetate buffer, wherein the pharmaceutical composition has a pH of about 4.5.

In specific embodiments, the aqueous pharmaceutical composition comprises:
about 1 mg/ml or about 10 mg/ml of the peptide, or pharmaceutically acceptable derivative thereof or pharmaceutically acceptable salt of any thereof, based on the amount of free peptide;
about 1 mg/ml or about 10 mg/ml of L-methionine, wherein the ratio of the concentration of methionine to the concentration of the peptide, or pharmaceutically acceptable derivative thereof or pharmaceutically acceptable salt of any thereof, is about 1:1, based on the amount of free peptide;
sorbitol as an osmolarity regulating agent;
about 0.2 mg/ml of benzalkonium chloride;
hydroxyethyl cellulose as a thickening agent, optionally at an amount that provides a viscosity of about 5 mPa·s or less; and
an acetate buffer, wherein the pharmaceutical composition has a pH of about 4.5.

In specific embodiments, the aqueous pharmaceutical composition comprises:
about 1 mg/ml or about 10 mg/ml of the peptide, or pharmaceutically acceptable derivative thereof or pharmaceutically acceptable salt of any thereof, based on the amount of free peptide;
about 1 mg/ml or about 10 mg/ml of L-methionine, wherein the ratio of the concentration of methionine to the concentration of the peptide, or pharmaceutically acceptable derivative thereof or pharmaceutically acceptable salt of any thereof, is about 1:1, based on the amount of free peptide;
sorbitol and sodium chloride as osmolarity regulating agents;
about 0.2 mg/ml of benzalkonium chloride;
hydroxyethyl cellulose as a thickening agent, optionally at an amount that provides a viscosity of about 5 mPa·s or less; and
an acetate buffer, wherein the pharmaceutical composition has a pH of about 4.5.

In specific embodiments, the aqueous pharmaceutical composition comprises:
about 1 mg/ml or about 10 mg/ml of the peptide, or pharmaceutically acceptable derivative thereof or pharmaceutically acceptable salt of any thereof, based on the amount of free peptide;
about 1 mg/ml or about 10 mg/ml of L-methionine, wherein the ratio of the concentration of methionine to the concentration of the peptide, or pharmaceutically acceptable derivative thereof or pharmaceutically acceptable salt of any thereof, is about 1:1, based on the amount of free peptide;
sodium chloride as an osmolarity regulating agent;
about 0.2 mg/ml of benzalkonium chloride;
hydroxypropyl methylcellulose as a thickening agent, optionally at an amount that provides a viscosity of about 5 mPa·s or less; and
an acetate buffer, wherein the pharmaceutical composition has a pH of about 4.5.

In specific embodiments, the aqueous pharmaceutical composition comprises:
about 1 mg/ml or about 10 mg/ml of the peptide, or pharmaceutically acceptable derivative thereof or pharmaceutically acceptable salt of any thereof, based on the amount of free peptide;
about 1 mg/ml or about 10 mg/ml of L-methionine, wherein the ratio of the concentration of methionine to the concentration of the peptide, or pharmaceutically acceptable derivative thereof or pharmaceutically acceptable salt of any thereof, is about 1:1, based on the amount of free peptide;
sorbitol as an osmolarity regulating agent;
about 0.2 mg/ml of benzalkonium chloride;
hydroxypropyl methylcellulose as a thickening agent, optionally at an amount that provides a viscosity of about 5 mPa·s or less; and
an acetate buffer, wherein the pharmaceutical composition has a pH of about 4.5.

In specific embodiments, the aqueous pharmaceutical composition comprises:
about 1 mg/ml or about 10 mg/ml of the peptide, or pharmaceutically acceptable derivative thereof or pharmaceutically acceptable salt of any thereof, based on the amount of free peptide;
about 1 mg/ml or about 10 mg/ml of L-methionine, wherein the ratio of the concentration of methionine to the concentration of the peptide, or pharmaceutically acceptable derivative thereof or pharmaceutically acceptable salt of any thereof, is about 1:1, based on the amount of free peptide;
sorbitol and sodium chloride as osmolarity regulating agents;
about 0.2 mg/ml of benzalkonium chloride;
hydroxypropyl methylcellulose as a thickening agent, optionally at an amount that provides a viscosity of about 5 mPa·s or less; and
an acetate buffer, wherein the pharmaceutical composition has a pH of about 4.5.

In specific embodiments, the aqueous pharmaceutical composition comprises:
about 1 mg/ml or about 10 mg/ml of the peptide, or pharmaceutically acceptable derivative thereof or pharmaceutically acceptable salt of any thereof, based on the amount of free peptide;
about 1 mg/ml or about 10 mg/ml of L-methionine, wherein the ratio of the concentration of methionine to the concentration of the peptide, or pharmaceutically acceptable derivative thereof or pharmaceutically acceptable salt of any thereof, is about 1:1, based on the amount of free peptide;
sodium chloride as an osmolarity regulating agent;
about 0.2 mg/ml of benzalkonium chloride;
hydroxyethyl cellulose as a thickening agent, optionally at an amount that provides a viscosity of about 5 mPa·s or less;
about 0.5 mg/ml of edetate disodium; and
an acetate buffer, wherein the pharmaceutical composition has a pH of about 4.5.

In specific embodiments, the aqueous pharmaceutical composition comprises: about 1 mg/ml or about 10 mg/ml of the peptide, or pharmaceutically acceptable derivative thereof or pharmaceutically acceptable salt of any thereof, based on the amount of free peptide;
about 1 mg/ml or about 10 mg/ml of L-methionine, wherein the ratio of the concentration of methionine to the concentration of the peptide, or pharmaceutically acceptable derivative thereof or pharmaceutically acceptable salt of any thereof, is about 1:1, based on the amount of free peptide;
sorbitol as an osmolarity regulating agent;
about 0.2 mg/ml of benzalkonium chloride;
hydroxyethyl cellulose as a thickening agent, optionally at an amount that provides a about 5 mPa·s or less;
about 0.5 mg/ml of edetate disodium; and
an acetate buffer, wherein the pharmaceutical composition has a pH of about 4.5.

In specific embodiments, the aqueous pharmaceutical composition comprises:
about 1 mg/ml or about 10 mg/ml of the peptide, or pharmaceutically acceptable derivative thereof or pharmaceutically acceptable salt of any thereof, based on the amount of free peptide;
about 1 mg/ml or about 10 mg/ml of L-methionine, wherein the ratio of the concentration of methionine to the concentration of the peptide, or pharmaceutically acceptable derivative thereof or pharmaceutically acceptable salt of any thereof, is about 1:1, based on the amount of free peptide;
sorbitol and sodium chloride as osmolarity regulating agents;
about 0.2 mg/ml of benzalkonium chloride;
hydroxyethyl cellulose as a thickening agent, optionally at an amount that provides a viscosity of about 5 mPa·s or less;
about 0.5 mg/ml of edetate disodium; and
an acetate buffer, wherein the pharmaceutical composition has a pH of about 4.5.

In specific embodiments, the aqueous pharmaceutical composition comprises:
about 1 mg/ml or about 10 mg/ml of the peptide, or pharmaceutically acceptable derivative thereof or pharmaceutically acceptable salt of any thereof, based on the amount of free peptide;
about 1 mg/ml or about 10 mg/ml of L-methionine, wherein the ratio of the concentration of methionine to the concentration of the peptide, or pharmaceutically acceptable derivative thereof or pharmaceutically acceptable salt of any thereof, is about 1:1, based on the amount of free peptide;
sodium chloride as an osmolarity regulating agent;
about 0.2 mg/ml of benzalkonium chloride;
hydroxypropyl methylcellulose as a thickening agent, optionally at an amount that provides a viscosity of about 5 mPa·s or less;
about 0.5 mg/ml of edetate disodium; and
an acetate buffer, wherein the pharmaceutical composition has a pH of about 4.5.

In specific embodiments, the aqueous pharmaceutical composition comprises:
about 1 mg/ml or about 10 mg/ml of the peptide, or pharmaceutically acceptable derivative thereof or pharmaceutically acceptable salt of any thereof, based on the amount of free peptide;
about 1 mg/ml or about 10 mg/ml of L-methionine, wherein the ratio of the concentration of methionine to the concentration of the peptide, or pharmaceutically acceptable derivative thereof or pharmaceutically acceptable salt of any thereof, is about 1:1, based on the amount of free peptide;
sorbitol as an osmolarity regulating agent;
about 0.2 mg/ml of benzalkonium chloride;
hydroxypropyl methylcellulose as a thickening agent, optionally at an amount that provides a viscosity of about 5 mPa·s or less;
about 0.5 mg/ml of edetate disodium; and
an acetate buffer, wherein the pharmaceutical composition has a pH of about 4.5.

In specific embodiments, the aqueous pharmaceutical composition comprises:
about 1 mg/ml or about 10 mg/ml of the peptide, or pharmaceutically acceptable derivative thereof or pharmaceutically acceptable salt of any thereof, based on the amount of free peptide;
about 1 mg/ml or about 10 mg/ml of L-methionine, wherein the ratio of the concentration of methionine to the concentration of the peptide, or pharmaceutically acceptable derivative thereof or pharmaceutically acceptable salt of any thereof, is about 1:1, based on the amount of free peptide;
sorbitol and sodium chloride as osmolarity regulating agents;
about 0.2 mg/ml of benzalkonium chloride;
hydroxypropyl methylcellulose as a thickening agent, optionally at an amount that provides a viscosity of about 5 mPa·s or less;
about 0.5 mg/ml of edetate disodium; and
an acetate buffer, wherein the pharmaceutical composition has a pH of about 4.5.

In specific embodiments, the aqueous pharmaceutical composition comprises:
about 1 mg/ml or about 10 mg/ml of the peptide, or pharmaceutically acceptable derivative thereof or pharmaceutically acceptable salt of any thereof, based on the amount of free peptide;
about 1 mg/ml or about 10 mg/ml of L-methionine, wherein the ratio of the concentration of methionine to the concentration of the peptide, or pharmaceutically acceptable derivative thereof or pharmaceutically acceptable salt of any thereof, is about 1:1, based on the amount of free peptide;
sorbitol as an osmolarity regulating agent;
about 0.2 mg/ml of benzalkonium chloride;
about 0.5 mg/ml of edetate disodium; and
an acetate buffer, wherein the pharmaceutical composition has a pH of about 4.5.

In specific embodiments, the aqueous pharmaceutical composition comprises:
about 1 mg/ml or about 10 mg/ml of the peptide, or pharmaceutically acceptable derivative thereof or pharmaceutically acceptable salt of any thereof, based on the amount of free peptide;
about 1 mg/ml or about 10 mg/ml of L-methionine, wherein the ratio of the concentration of methionine to the concentration of the peptide, or pharmaceutically acceptable derivative thereof or pharmaceutically acceptable salt of any thereof, is about 1:1, based on the amount of free peptide;
sorbitol as an osmolarity regulating agent;
about 0.2 mg/ml of benzalkonium chloride; and
about 0.5 mg/ml of edetate disodium.

In specific embodiments, the aqueous pharmaceutical composition comprises:
about 1 mg/ml or about 10 mg/ml of the peptide, or pharmaceutically acceptable derivative thereof or pharmaceutically acceptable salt of any thereof, based on the amount of free peptide;
about 1 mg/ml or about 10 mg/ml of L-methionine, wherein the ratio of the concentration of methionine to the concentration of the peptide, or pharmaceutically acceptable derivative thereof or pharmaceutically acceptable salt of any thereof, is about 1:1, based on the amount of free peptide;
sodium chloride as an osmolarity regulating agent;
about 0.2 mg/ml of benzalkonium chloride;
hydroxypropyl methylcellulose as a thickening agent, optionally at an amount that provides a viscosity of about 5 mPa·s or less; and
about 0.5 mg/ml of edetate disodium.

In specific embodiments, the aqueous pharmaceutical composition comprises:
about 1 mg/ml or about 10 mg/ml of the peptide, or pharmaceutically acceptable derivative thereof or pharmaceutically acceptable salt of any thereof, based on the amount of free peptide;
about 1 mg/ml or about 10 mg/ml of L-methionine, wherein the ratio of the concentration of methionine to the concentration of the peptide, or pharmaceutically acceptable derivative thereof or pharmaceutically acceptable salt of any thereof, is about 1:1, based on the amount of free peptide;
sorbitol as an osmolarity regulating agent;
about 0.2 mg/ml of benzalkonium chloride;
hydroxypropyl methylcellulose as a thickening agent, optionally at an amount that provides a viscosity of about 5 mPa·s or less; and
about 0.5 mg/ml of edetate disodium.

In specific embodiments, the aqueous pharmaceutical composition comprises:
about 1 mg/ml or about 10 mg/ml of the peptide, or pharmaceutically acceptable derivative thereof or pharmaceutically acceptable salt of any thereof, based on the amount of free peptide;
about 1 mg/ml or about 10 mg/ml of L-methionine, wherein the ratio of the concentration of methionine to the concentration of the peptide, or pharmaceutically acceptable derivative thereof or pharmaceutically acceptable salt of any thereof, is about 1:1, based on the amount of free peptide;
sorbitol and sodium chloride as osmolarity regulating agents;
about 0.2 mg/ml of benzalkonium chloride;
hydroxypropyl methylcellulose as a thickening agent, optionally at an amount that provides a viscosity of about 5 mPa·s or less; and
about 0.5 mg/ml of edetate disodium.

In specific embodiments, the aqueous pharmaceutical composition comprises:
about 1 mg/ml or about 10 mg/ml of the peptide, or pharmaceutically acceptable derivative thereof or pharmaceutically acceptable salt of any thereof, based on the amount of free peptide;
about 1 mg/ml or about 10 mg/ml of L-methionine, wherein the ratio of the concentration of methionine to the concentration of the peptide, or pharmaceutically acceptable derivative thereof or pharmaceutically acceptable salt of any thereof, is about 1:1, based on the amount of free peptide;
sodium chloride as an osmolarity regulating agent;
about 0.2 mg/ml of benzalkonium chloride; and
hydroxypropyl methylcellulose as a thickening agent, optionally at an amount that provides a viscosity of about 5 mPa·s or less.

In specific embodiments, the aqueous pharmaceutical composition comprises:
about 1 mg/ml or about 10 mg/ml of the peptide, or pharmaceutically acceptable derivative thereof or pharmaceutically acceptable salt of any thereof, based on the amount of free peptide;
about 1 mg/ml or about 10 mg/ml of L-methionine, wherein the ratio of the concentration of methionine to the concentration of the peptide, or pharmaceutically acceptable derivative thereof or pharmaceutically acceptable salt of any thereof, is about 1:1, based on the amount of free peptide;
sorbitol as an osmolarity regulating agent;
about 0.2 mg/ml of benzalkonium chloride; and
hydroxypropyl methylcellulose as a thickening agent, optionally at an amount that provides a viscosity of about 5 mPa·s or less.

In specific embodiments, the aqueous pharmaceutical composition comprises:
about 1 mg/ml or about 10 mg/ml of the peptide, or pharmaceutically acceptable derivative thereof or pharmaceutically acceptable salt of any thereof, based on the amount of free peptide;
about 1 mg/ml or about 10 mg/ml of L-methionine, wherein the ratio of the concentration of methionine to the concentration of the peptide, or pharmaceutically acceptable derivative thereof or pharmaceutically acceptable salt of any thereof, is about 1:1, based on the amount of free peptide;
sorbitol and sodium chloride as osmolarity regulating agents;
about 0.2 mg/ml of benzalkonium chloride; and
hydroxypropyl methylcellulose as a thickening agent, optionally at an amount that provides a viscosity of about 5 mPa·s or less.

In accordance with any of the foregoing embodiments, the composition may comprise an acetate salt of the peptide. Alternatively, the composition may comprise a pharmaceutically acceptable derivative of the peptide having an N-terminal acetyl moiety. Alternatively, the composition may comprise a pharmaceutically acceptable derivative of the peptide having a C-terminal amidate moiety or a C-terminal adamantane moiety. Alternatively, the composition may comprise a pharmaceutically acceptable derivative of the peptide having an N-terminal acetyl moiety and a C-terminal amidate moiety or a C-terminal adamantane moiety. In accordance with any of the foregoing embodiments, the composition may comprise a pharmaceutically acceptable derivative of the peptide selected from:
N-acetyl-Met-Glu-His-Phe-Pro-Gly-Pro;
N-acetyl-Met-Glu-His-Phe-Pro-Gly-Pro-amidate, and
N-acetyl-Met-Glu-His-Phe-Pro-Gly-Pro-adamantane-CONH₂.

### Therapeutic Methods/Uses

The present disclosure also provides therapeutic uses of the compositions described herein in treatment methods that comprise administering a therapeutically effective amount of a composition as described herein to a subject in need thereof by intranasal administration. The therapeutic uses may be for any use currently indicated or proposed for the peptide, including, but not limited to, treating one or more of intellectual-mental disorders in vascular malformations of the brain, dyscirculatory encephalopathy, transient ischemic attack (TIA), neurotic disorders, glaucoma, optic nerve disorders, minimal brain dysfunctions in children, including attention deficit hyperactivity disorder (ADHD), and mental fatigue, multiple sclerosis, perinatal damage of CNS with atonic-astatic syndrome, and organic mental disorders in children, or for promoting recovery after craniocerebral injury or promoting recovery after narcosis, or for stroke management or treatment after neurosurgery.

In some embodiments, a composition as described herein is packaged with or provided in and administered from, a nasal spray drug delivery device. Nasal spray drug delivery devices suitable for administration of a composition as described herein are not particularly limited, and include nasal spray drug delivery devices that are known in the art and commercially available. Examples of suitable nasal spray drug delivery devices include but are not limited to those that use the CPS Technology Platform available from Aptar Pharma and SP270+ or SP370+ devices available from Nemera. Use of a nasal spray drug delivery device may promote nose-to-brain delivery of the peptide via the olfactory region. Furthermore, use of a nasal spray drug delivery device reduces the likelihood of a portion of the administered dose dripping out of the nasal cavity, which is a problem with the SEMAX product that can result in delivery of less than the full dose.

In advantageous embodiments, the nasal spray drug delivery device is configured to provide accurate and consisting dosing of an exact amount of formulation per spray, in accordance with requirements of the U.S. Pharmacopoeia, European Pharmacopoeia, Russia Pharmacopoeia, and/or the Pharmacopoeia of EAEC, such as a target volume of about 100 μL per spray. The nasal spray drug delivery device may be configured to provide a non-inhalable droplet size of the composition (such as a droplet size of more than 10 μm). The nasal spray drug delivery device may be configured to provide a narrowed spraying angle and a decreased spray pattern area that focus delivery to the upper regions of the nasal cavity. The nasal spray drug delivery device may be configured to provide fine droplets that distribute the composition in nasal cavity evenly, which can contribute to better absorption. The nasal spray drug delivery device may be configured to permit fast priming. The nasal spray drug delivery device may be configured to have no need for re-priming. The nasal spray drug delivery device may be configured to reduce or prevent metal-induced oxidation of the peptide, such as by having a metal-free fluid path. The nasal spray drug delivery device may be configured to have a reduced incidence of clogging or crystallization, such as by having a reinforced tip sealing mechanism. The nasal spray drug delivery device may be configured to spray at any angle, which may be important for specific indications where the subject is not able to maintain an upright position, such as stroke management. The nasal spray drug delivery device may be configured to have microbial integrity, such as by having an advanced filter membrane.

The peptide compositions described herein may be formulated to provide a therapeutically effective dose in a suitable volume (such as about 100 μL), in one or more sprays into one or both nostrils. Thus, a therapeutic use as described herein may comprise administering a composition as described herein to a subject in need thereof, by intranasal administration of one or more sprays into one or both nostrils, using a suitable nasal spray drug delivery device.

### EXAMPLES

The following specific examples are included as illustrative examples of the compositions described herein. These examples are in no way intended to limit the scope of the disclosure. Other aspects of the disclosure will be apparent to those skilled in the art to which the disclosure pertains.

In the examples that follow, the acetate salt of the peptide was used to make the test formulations and exemplary embodiments of Table 1 and Table 2. The reported amounts of peptide refer to the amount of free peptide present (acetate-free, anhydrous).

### Example 1

The effect of methionine on the stability of the peptide in an aqueous composition was evaluated. Test formulations of the peptide (1 mg/ml) in aqueous acetate buffer at pH 5.5 were prepared with or without methionine (1 mg/ml). After storage for 14 days at 60° C., the formulations were evaluated for changes in content of various impurities by UPLC analysis (column: ACQUITY UPLC® BEH C18 50×2.1 (1.7 mcm); gradient mode; flow rate: 0.3 ml/min; detection wavelength: 210 nm; column temperature: 40° C.). The results reported below show that the methionine improved stability and inhibited the formation of various impurities.

| | | Peptide + |
|---|---|---|
| Composition | Peptide | methionine |
| Impurity (identified by retention time (min.)) | Δ* | Δ* |
| 1.293 | 1.19 | 1.13 |
| 4.923 | 5.58 | 5.49 |
| 5.821 | 0.1 | 0 |
| 6.267 | 0.27 | 0.25 |
| 7.268 | 0.31 | 0.11 |
| 10.47 | 0.84 | 0.82 |
| Total | 8.49 | 7.99 |

*Difference in % (w/w) of impurities from Day 0 to Day 14.

### Example 2

The effect of pH on the stability of the peptide in an aqueous composition was evaluated. Test formulations of the peptide in water (1 mg/ml) were prepared at a pH of 4.5, 5.0 or 5.5 using an acetate buffer. After storage for 14 days at 60° C., the formulations were evaluated for changes in content of various impurities by UPLC analysis as described above. The results reported in the below table show that the peptide is most stable at pH 4.5, as reflected in reduced formation of various impurities.

| pH | 4.5 | 5.0 | 5.5 |
|---|---|---|---|
| impurity (identified by retention time (min.)) | Δ* | Δ* | Δ* |
| 1.293 | 0.3 | 0.55 | 1.19 |
| 4.923 | 1.43 | 2.58 | 5.58 |
| 5.821 | 0.28 | 0.12 | 0.1 |
| 6.267 | 0 | 0.11 | 0.27 |
| 7.268 | 0.28 | 0.28 | 0.31 |
| 10.47 | 0.1 | 0.24 | 0.84 |
| Total | 2.55 | 4.07 | 8.49 |

*Difference in % (w/w) of impurities from Day 0 to Day 14.

### Example 3

The effect of an osmolarity regulating agent, such as sorbitol, on the stability of the peptide in an aqueous composition was evaluated. Test formulations of the peptide (1 mg/ml) in aqueous acetate buffer at pH 5.5 were prepared with or without sorbitol (55 mg/ml). After storage for 14 days at 60° C., the formulations were evaluated for changes in content of various impurities by UPLC as described above. The results reported in the below table show that sorbitol improved stability and inhibited the formation of various impurities.

| Composition | Peptide | Peptide + Sorbitol |
|---|---|---|
| impurity (identified by retention time (min.)) | Δ* | Δ* |
| 1.293 | 1.19 | 1.09 |
| 4.923 | 5.58 | 5.04 |
| 5.821 | 0.1 | 0 |
| 6.267 | 0.27 | 0.25 |
| 7.268 | 0.31 | 0.29 |
| 10.47 | 0.84 | 0.83 |
| Total | 8.49 | 7.7 |

*Difference in % (w/w) of impurities from Day 0 to Day 14.

### Example 4

The effect of including a chelating agent, such as EDTA, on the stability of peptide compositions as described herein was evaluated. Unbuffered test formulations of the peptide were prepared with the components listed in the table below. After storage for 7 days at 60° C., the formulations were evaluated for changes in pH. The results reported in the below table show that including EDTA improves stability, as reflected in the more stable pH of EDTA-containing compositions.

| Composition | pH at Day 0 | pH at Day 7 |
|---|---|---|
| 7.1 mg/ml NaCl, 1 mg/ml Methionine, | 5.1 | 6.0 |
| 0.2 mg/ml BAC* | | |
| 43 mg/ml sorbitol, 1 mg/ml Methionine, | 4.9 | 5.3 |
| 0.2 mg/ml BAC | | |
| 3 mg/ml NaCl, 20.8 mg/ml sorbitol, | 4.9 | 5.4 |
| 1 mg/ml Methionine, 0.2 mg/ml BAC | | |
| 7.1 mg/ml NaCl, 1 mg/ml Methionine, | 4.6 | 4.8 |
| 0.2 mg/ml BAC, 0.5 mg/ml EDTA | | |
| 43 mg/ml sorbitol, 1 mg/ml Methionine, | 4.8 | 4.9 |
| 0.2 mg/ml BAC, 0.5 mg/ml EDTA | | |
| 3 mg/ml NaCl, 20.8 mg/ml sorbitol, | 4.7 T | 4.8 |
| 1 mg/ml Methionine, 0.2 mg/ml BAC, | | |
| 0.5 mg/ml EDTA | | |

*BAC = benzalkonium chloride

### Example 5

Formulations A1-A10 and B1-B10 are specific examples of compositions as disclosed herein. The compositions have a viscosity of 5 mPa·s or less.

TABLE 1

| | Formulation No | | | | | | | | | |
|---|---|---|---|---|---|---|---|---|---|---|
| | A1 | A2 | A3 | A4 | A5 | A6 | A7 | A8 | A9 | A10 |
| Component | Concentration mg/ml | | | | | | | | | |
| Peptide | 1 or 10 | 1 or 10 | 1 or 10 | 1 or 10 | 1 or 10 | 1 or 10 | 1 or 10 | 1 or 10 | 1 or 10 | 1 or 10 |
| sodium acetate | To pH | To pH | To pH | To pH | To pH | To pH | To pH | To pH | To pH | To pH |
| 3-hydrate | 4.5 | 4.5 | 4.5 | 4.5 | 4.5 | 4.5 | 4.5 | 4.5 | 4.5 | 4.5 |
| acetic acid | To pH | To pH | To pH | To pH | To pH | To pH | To pH | To pH | To pH | To pH |
| (glacial) | 4.5 | 4.5 | 4.5 | 4.5 | 4.5 | 4.5 | 4.5 | 4.5 | 4.5 | 4.5 |
| L-methionine | 1 or 10 | 1 or 10 | 1 or 10 | 1 or 10 | 1 or 10 | 1 or 10 | 1 or 10 | 1 or 10 | 1 or 10 | 1 or 10 |
| (antioxidant) | | | | | | | | | | |
| sorbitol | — | q.s. to | q.s. to | — | q.s. to | q.s. to | — | q.s. to | q.s. to | — |
| (osmolarity | | isotonic | isotonic | | isotonic | isotonic | | isotonic | isotonic | |
| regulating agent) | | solution | solution | | solution | solution | | solution | solution | |
| sodium chloride | q.s. to | — | q.s. to | q.s. to | — | q.s. to | q.s. to | — | q.s. to | q.s. to |
| (osmolarity | isotonic | | isotonic | isotonic | | isotonic | isotonic | | isotonic | isotonic |
| regulating agent) | solution | | solution | solution | | solution | solution | | solution | solution |
| benzalkonium | 0.2 | 0.2 | 0.2 | 0.2 | 0.2 | 0.2 | 0.2 | 0.2 | 0.2 | 0.2 |
| chloride | | | | | | | | | | |
| (antimicrobial | | | | | | | | | | |
| preservative) | | | | | | | | | | |
| HPMC | — | — | — | 1 | 1 | 1 | — | — | — | 1 |
| (thickening agent) | | | | | | | | | | |
| HEC | 1 | 1 | 1 | — | — | — | 1 | 1 | 1 | — |
| (thickening agent) | | | | | | | | | | |
| edetate disodium | — | — | — | — | — | — | 0.5 | 0.5 | 0.5 | 0.5 |
| (chelating agent) | | | | | | | | | | |
| Water (purified) | q.s. | q.s. | q.s. | q.s. | q.s. | q.s. | q.s. | q.s. | q.s. | q.s. |
| | 1 ml | 1 ml | 1 ml | 1 ml | 1 ml | 1 ml | 1 ml | 1 ml | 1 ml | 1 ml |

TABLE 2

| | Formulation No | | | | | | | | | |
|---|---|---|---|---|---|---|---|---|---|---|
| | B1 | B2 | B3 | B4 | B5 | B6 | B7 | B8 | B9 | B10 |
| Component | Concentration mg/ml | | | | | | | | | |
| Peptide | 1 or 10 | 1 or 10 | 1 or 10 | 1 or 10 | 1 or 10 | 1 or 10 | 1 or 10 | 1 or 10 | 1 or 10 | 1 or 10 |
| sodium acetate | To pH | To pH | To pH | | | | | | | |
| 3-hydrate | 4.5 | 4.5 | 4.5 | — | — | — | — | — | — | — |
| acetic acid | To pH | To pH | To pH | | | | | | | |
| (glacial) | 4.5 | 4.5 | 4.5 | — | — | — | — | — | — | — |
| L- methionine | 1 or 10 | 1 or 10 | 1 or 10 | 1 or 10 | 1 or 10 | 1 or 10 | 1 or 10 | 1 or 10 | 1 or 10 | 1 or 10 |
| (antioxidant) | | | | | | | | | | |
| sorbitol | q.s. to | q.s. to | q.s. to | q.s. to | — | q.s. to | q.s. to | — | q.s. to | q.s. to |
| (osmolarity | isotonic | isotonic | isotonic | isotonic | | isotonic | isotonic | | isotonic | isotonic |
| regulating agent) | solution | solution | solution | solution | | solution | solution | | solution | solution |
| sodium chloride | — | q.s. to | — | — | q.s. to | — | q.s. to | q.s. to | — | q.s. to |
| (osmolarity | | isotonic | | | isotonic | | isotonic | isotonic | | isotonic |
| regulating agent) | | solution | | | solution | | solution | solution | | solution |
| benzalkonium | 0.2 | 0.2 | 0.2 | 0.2 | 0.2 | 0.2 | 0.2 | 0.2 | 0.2 | 0.2 |
| chloride | | | | | | | | | | |
| (antimicrobial | | | | | | | | | | |
| preservative) | | | | | | | | | | |
| HPMC | 1 | 1 | — | — | 1 | 1 | 1 | 1 | 1 | 1 |
| (thickening agent) | | | | | | | | | | |
| edetate disodium | 0.5 | 0.5 | 0.5 | 0.5 | 0.5 | 0.5 | 0.5 | — | — | — |
| (chelating agent) | | | | | | | | | | |
| Water purified | q.s. | q.s. | q.s. | q.s. | q.s. | q.s. | q.s. | q.s. | q.s. | q.s. |
| | 1 ml | 1 ml | 1 ml | 1 ml | 1 ml | 1 ml | 1 ml | 1 ml | 1 ml | 1 ml |

What is claimed is:

1. An aqueous pharmaceutical composition comprising:
(a) peptide Met-Glu-His-Phe-Pro-Gly-Pro (SEQ ID NO: 1), or a pharmaceutically acceptable derivative thereof or a pharmaceutically acceptable salt of any thereof;
(b) an antioxidant;
(c) an osmolarity regulating agent;
(d) optionally, an antimicrobial preservative; and
(e) water,
wherein the pharmaceutical composition has a pH of from about 4.0 to about 7.0, and an osmolarity of from about 235 mOsm/L to about 380 mOsm/L.

2. The pharmaceutical composition of claim 1, further comprising a buffering agent.

3. The pharmaceutical composition of claim 2, wherein the buffering agent provides a buffer selected from one or more of an acetate buffer, a citrate buffer, a phosphate buffer, a phosphate-citrate buffer, and a succinate buffer.

4. The pharmaceutical composition of claim 2, wherein the buffering agent provides an acetate buffer.

5. The pharmaceutical composition of any one the preceding claims, wherein the pharmaceutical composition has a pH of from about 4.5 to about 5.0.

6. The pharmaceutical composition of any one the preceding claims, wherein the pharmaceutical composition has a pH of from about 4.0 to about 6.5.

7. The pharmaceutical composition of any one the preceding claims, wherein the pharmaceutical composition has a pH of from about 4.0 to about 5.5.

8. The pharmaceutical composition of any one the preceding claims, wherein the pharmaceutical composition has a pH of about 4.5.

9. The pharmaceutical composition of any one of the preceding claims, wherein the peptide, pharmaceutically acceptable derivative thereof, or pharmaceutically acceptable salt of any thereof, is present at a concentration of from about 0.1 mg/ml to about 100 mg/ml, based on the free peptide.

10. The pharmaceutical composition of any one of the preceding claims, wherein the peptide, pharmaceutically acceptable derivative thereof, or pharmaceutically acceptable salt of any thereof, is present at a concentration of from about 0.5 mg/ml to about 50 mg/ml, based on the free peptide.

11. The pharmaceutical composition of any one of the preceding claims, wherein the peptide, pharmaceutically acceptable derivative thereof, or pharmaceutically acceptable salt of any thereof, is present at a concentration of from about 0.8 mg/ml to about 12 mg/ml, based on the free peptide.

12. The pharmaceutical composition of any one of the preceding claims, wherein the antioxidant is one or more selected from an amino acid, a vitamin, a vitamin derivative, an inorganic salt, and a phenol.

13. The pharmaceutical composition of any one of the preceding claims, wherein the antioxidant comprises an amino acid selected from methionine, cysteine, histidine, asparagine, glycine, alanine, valine, phenylalanine, and cystine, optionally wherein the amino acid is the L-form.

14. The pharmaceutical composition of any one of the preceding claims, wherein the antioxidant comprises methionine, optionally wherein the methionine is the L-form.

15. The pharmaceutical composition of any one of the preceding claims, wherein the antioxidant comprises ascorbic acid or a derivative thereof.

16. The pharmaceutical composition of any one of the preceding claims, wherein the antioxidant comprises an inorganic salt selected from sodium thiosulfate, sodium metabisulfite, and potassium metabisulfite.

17. The pharmaceutical composition of any one of the preceding claims, wherein the antioxidant comprises a phenol selected from thymol and hydroxyanisole.

18. The pharmaceutical composition of any one of the preceding claims, wherein the antioxidant is present at a concentration ratio of antioxidant to peptide or pharmaceutically acceptable derivative thereof or pharmaceutically acceptable salt of any thereof, of from about 0.1:1 to about 10:1, based on the total amount of antioxidant present and the amount of free peptide.

19. The pharmaceutical composition of any one of the preceding claims, wherein the antioxidant is present at a concentration ratio of antioxidant to peptide or pharmaceutically acceptable derivative thereof or pharmaceutically acceptable salt of any thereof, of from about 0.1:1 to about 5:1, based on the total amount of antioxidant present and the amount of free peptide.

20. The pharmaceutical composition of any one of the preceding claims, wherein the antioxidant is present at a concentration ratio of antioxidant to peptide or pharmaceutically acceptable derivative thereof or pharmaceutically acceptable salt of any thereof, of about 1:1, based on the total amount of antioxidant present and the amount of free peptide.

21. The pharmaceutical composition of any one of the preceding claims, wherein the antimicrobial preservative is present and is one or more selected from a quaternary ammonium compound, sorbic acid, glycerol, benzyl alcohol, chlorobutanol, phenyl ethanol, and benzoic acid or a salt thereof.

22. The pharmaceutical composition of any one of the preceding claims, wherein the antimicrobial preservative comprises the quaternary ammonium compound benzalkonium chloride.

23. The pharmaceutical composition of any one of the preceding claims, wherein the antimicrobial preservative is present at a concentration of from about 0.01 mg/ml to about 2 mg/ml.

24. The pharmaceutical composition of any one of the preceding claims, wherein the antimicrobial preservative is present at a concentration of from about 0.02 mg/ml to about 1.2 mg/ml.

25. The pharmaceutical composition of any one of the preceding claims, wherein the osmolarity regulating agent is one or more selected from an inorganic salt and a polyol.

26. The pharmaceutical composition of any one of the preceding claims, wherein the osmolarity regulating agent comprises one or more selected from sorbitol, glycerol, mannitol, maltitol, dextrose, and sucrose.

27. The pharmaceutical composition of any one of the preceding claims, wherein the osmolarity regulating agent comprises an inorganic salt selected from sodium chloride and sodium sulfate.

28. The pharmaceutical composition of any one of the preceding claims, wherein the osmolarity regulating agent comprises sodium chloride and sorbitol.

29. The pharmaceutical composition of any one of the preceding claims, wherein the osmolarity regulating agent comprises glycerol.

30. The pharmaceutical composition of any one of the preceding claims, wherein the osmolarity regulating agent is present at a concentration of from about 0.01 mg/ml to about 100 mg/ml.

31. The pharmaceutical composition of any one of the preceding claims, further comprising a chelating agent.

32. The pharmaceutical composition of claim 31, wherein the chelating agent is one or more selected from a tetrabasic carboxylic acid, a tribasic carboxylic acid, a dibasic carboxylic acid, a dibasic hydroxycarboxylic acid, and a pharmaceutically acceptable salt thereof.

33. The pharmaceutical composition of claim 31, wherein the chelating agent comprises edetate disodium (EDTA).

34. The pharmaceutical composition of any one of claims 31-33, wherein the chelating agent is present at a concentration of from about 0.01 mg/ml to about 5 mg/ml.

35. The pharmaceutical composition of any one of claims 31-33, wherein the chelating agent is present at a concentration of from about 0.3 mg/ml to about 2 mg/ml.

36. The pharmaceutical composition of any one of the preceding claims, further comprising a thickening agent.

37. The pharmaceutical composition of claim 36, wherein the thickening agent is one or more selected from cellulose and cellulose derivatives (optionally hydroxyethyl cellulose, hydroxypropyl methylcellulose, microcrystalline cellulose, and/or carboxymethylcellulose), polyvinylpyrrolidone, acrylic acid polymers and copolymers, polyoxyethylene-polyoxypropylene block copolymers, and salts of any thereof.

38. The pharmaceutical composition of any one of claims 36-37, wherein the thickening agent is present at a concentration of from about of about 0.1 mg/ml to about 100 mg/ml.

39. The pharmaceutical composition of any one of claims 36-37, wherein the thickening agent is present at a concentration of from about 0.5 mg/ml to about 5 mg/ml.

40. The pharmaceutical composition of any one of the preceding claims, wherein the composition has a viscosity of about 5 mPa·s or less.

41. The pharmaceutical composition of any one of the preceding claims, wherein:
the antioxidant comprises L-methionine;
the osmolarity regulating agent comprises one or both of sorbitol and sodium chloride;
the antimicrobial preservative is present and comprises benzalkonium chloride;
if present, the thickening agent comprises one or both of hydroxypropyl methylcellulose and hydroxyethyl cellulose;
if present, the chelating agent comprises edetate disodium; and
if present, the buffering agent comprises an acetate buffer.

42. The pharmaceutical composition of claim 1, comprising:
about 1 mg/ml or about 10 mg/ml of the peptide, or pharmaceutically acceptable derivative thereof, or pharmaceutically acceptable salt of any thereof, based on the amount of free peptide;
about 1 mg/ml or about 10 mg/ml of L-methionine, wherein the ratio of the concentration of methionine to the concentration of the peptide or pharmaceutically acceptable derivative thereof or pharmaceutically acceptable salt of any thereof, is about 1:1, based on the amount of free peptide;
sodium chloride as an osmolarity regulating agent;
about 0.2 mg/ml of benzalkonium chloride;
hydroxyethyl cellulose as a thickening agent, optionally in an amount that provides a viscosity of about 5 mPa·s or less; and
an acetate buffer, wherein the pharmaceutical composition has a pH of about 4.5.

43. The pharmaceutical composition of claim 1, comprising:
about 1 mg/ml or about 10 mg/ml of the peptide, or pharmaceutically acceptable derivative thereof, or pharmaceutically acceptable salt of any thereof, based on the amount of free peptide;
about 1 mg/ml or about 10 mg/ml of L-methionine, wherein the ratio of the concentration of methionine to the concentration of the peptide or pharmaceutically acceptable derivative thereof or pharmaceutically acceptable salt of any thereof, is about 1:1, based on the amount of free peptide;
sorbitol as an osmolarity regulating agent;
about 0.2 mg/ml of benzalkonium chloride;
hydroxyethyl cellulose as a thickening agent, optionally in an amount that provides a viscosity of about 5 mPa·s or less; and
an acetate buffer, wherein the pharmaceutical composition has a pH of about 4.5.

44. The pharmaceutical composition of claim 1, comprising:
about 1 mg/ml or about 10 mg/ml of the peptide, or pharmaceutically acceptable derivative thereof, or pharmaceutically acceptable salt of any thereof, based on the amount of free peptide;
about 1 mg/ml or about 10 mg/ml of L-methionine, wherein the ratio of the concentration of methionine to the concentration of the peptide or pharmaceutically acceptable derivative thereof or pharmaceutically acceptable salt of any thereof, is about 1:1, based on the amount of free peptide;
sorbitol and sodium chloride as osmolarity regulating agents;
about 0.2 mg/ml of benzalkonium chloride;
hydroxyethyl cellulose as a thickening agent, optionally in an amount that provides a viscosity of about 5 mPa·s or less; and
an acetate buffer, wherein the pharmaceutical composition has a pH of about 4.5.

45. The pharmaceutical composition of claim 1, comprising:
about 1 mg/ml or about 10 mg/ml of the peptide, or pharmaceutically acceptable derivative thereof, or pharmaceutically acceptable salt of any thereof, based on the amount of free peptide;
about 1 mg/ml or about 10 mg/ml of L-methionine, wherein the ratio of the concentration of methionine to the concentration of the peptide or pharmaceutically acceptable derivative thereof or pharmaceutically acceptable salt of any thereof, is about 1:1, based on the amount of free peptide;
sodium chloride as an osmolarity regulating agent;
about 0.2 mg/ml of benzalkonium chloride;
hydroxypropyl methylcellulose as a thickening agent, optionally in an amount that provides a viscosity of about 5 mPa·s or less; and
an acetate buffer, wherein the pharmaceutical composition has a pH of about 4.5.

46. The pharmaceutical composition of claim 1, comprising:
about 1 mg/ml or about 10 mg/ml of the peptide, or pharmaceutically acceptable derivative thereof, or pharmaceutically acceptable salt of any thereof, based on the amount of free peptide;
about 1 mg/ml or about 10 mg/ml of L-methionine, wherein the ratio of the concentration of methionine to the concentration of the peptide or pharmaceutically acceptable derivative thereof or pharmaceutically acceptable salt of any thereof, is about 1:1, based on the amount of free peptide;
sorbitol as an osmolarity regulating agent;
about 0.2 mg/ml of benzalkonium chloride;
hydroxypropyl methylcellulose as a thickening agent, optionally in an amount that provides a viscosity of about 5 mPa·s or less; and
an acetate buffer, wherein the pharmaceutical composition has a pH of about 4.5.

47. The pharmaceutical composition of claim 1, comprising:
about 1 mg/ml or about 10 mg/ml of the peptide, or pharmaceutically acceptable derivative thereof, or pharmaceutically acceptable salt of any thereof, based on the amount of free peptide;
about 1 mg/ml or about 10 mg/ml of L-methionine, wherein the ratio of the concentration of methionine to the concentration of the peptide or pharmaceutically acceptable derivative thereof or pharmaceutically acceptable salt of any thereof, is about 1:1, based on the amount of free peptide;
sorbitol and sodium chloride as osmolarity regulating agents;
about 0.2 mg/ml of benzalkonium chloride;
hydroxypropyl methylcellulose as a thickening agent, optionally in an amount that provides a viscosity of about 5 mPa·s or less; and
an acetate buffer, wherein the pharmaceutical composition has a pH of about 4.5.

48. The pharmaceutical composition of claim 1, comprising:
about 1 mg/ml or about 10 mg/ml of the peptide, or pharmaceutically acceptable derivative thereof, or pharmaceutically acceptable salt of any thereof, based on the amount of free peptide;
about 1 mg/ml or about 10 mg/ml of L-methionine, wherein the ratio of the concentration of methionine to the concentration of the peptide or pharmaceutically acceptable derivative thereof or pharmaceutically acceptable salt of any thereof, is about 1:1, based on the amount of free peptide;
sodium chloride as an osmolarity regulating agent;
about 0.2 mg/ml of benzalkonium chloride;
hydroxyethyl cellulose as a thickening agent, optionally in an amount that provides a viscosity of about 5 mPa·s or less;
about 0.5 mg/ml of edetate disodium; and
an acetate buffer, wherein the pharmaceutical composition has a pH of about 4.5.

49. The pharmaceutical composition of claim 1, comprising:
about 1 mg/ml or about 10 mg/ml of the peptide, or pharmaceutically acceptable derivative thereof, or pharmaceutically acceptable salt of any thereof, based on the amount of free peptide;
about 1 mg/ml or about 10 mg/ml of L-methionine, wherein the ratio of the concentration of methionine to the concentration of the peptide or pharmaceutically acceptable derivative thereof or pharmaceutically acceptable salt of any thereof, is about 1:1, based on the amount of free peptide;
sorbitol as an osmolarity regulating agent;
about 0.2 mg/ml of benzalkonium chloride;
hydroxyethyl cellulose as a thickening agent, optionally in an amount that provides a viscosity of about 5 mPa·s or less;
about 0.5 mg/ml of edetate disodium; and
an acetate buffer, wherein the pharmaceutical composition has a pH of about 4.5.

50. The pharmaceutical composition of claim 1, comprising:
about 1 mg/ml or about 10 mg/ml of the peptide, or pharmaceutically acceptable derivative thereof, or pharmaceutically acceptable salt of any thereof, based on the amount of free peptide;
about 1 mg/ml or about 10 mg/ml of L-methionine, wherein the ratio of the concentration of methionine to the concentration of the peptide or pharmaceutically acceptable derivative thereof or pharmaceutically acceptable salt of any thereof, is about 1:1, based on the amount of free peptide;
sorbitol and sodium chloride as osmolarity regulating agents;
about 0.2 mg/ml of benzalkonium chloride;
hydroxyethyl cellulose as a thickening agent, optionally in an amount that provides a viscosity of about 5 mPa·s or less;
about 0.5 mg/ml of edetate disodium; and
an acetate buffer, wherein the pharmaceutical composition has a pH of about 4.5.

51. The pharmaceutical composition of claim 1, comprising:
about 1 mg/ml or about 10 mg/ml of the peptide, or pharmaceutically acceptable derivative thereof, or pharmaceutically acceptable salt of any thereof, based on the amount of free peptide;
about 1 mg/ml or about 10 mg/ml of L-methionine, wherein the ratio of the concentration of methionine to the concentration of the peptide or pharmaceutically acceptable derivative thereof or pharmaceutically acceptable salt of any thereof, is about 1:1, based on the amount of free peptide;
sodium chloride as an osmolarity regulating agent;
about 0.2 mg/ml of benzalkonium chloride;
hydroxypropyl methylcellulose as a thickening agent, optionally in an amount that provides a viscosity of about 5 mPa·s or less;
about 0.5 mg/ml of edetate disodium; and
an acetate buffer, wherein the pharmaceutical composition has a pH of about 4.5.

52. The pharmaceutical composition of claim 1, comprising:
about 1 mg/ml or about 10 mg/ml of the peptide, or pharmaceutically acceptable derivative thereof, or pharmaceutically acceptable salt of any thereof, based on the amount of free peptide;
about 1 mg/ml or about 10 mg/ml of L-methionine, wherein the ratio of the concentration of methionine to the concentration of the peptide or pharmaceutically acceptable derivative thereof or pharmaceutically acceptable salt of any thereof, is about 1:1, based on the amount of free peptide;
sorbitol as an osmolarity regulating agent;
about 0.2 mg/ml of benzalkonium chloride;
hydroxypropyl methylcellulose as a thickening agent, optionally in an amount that provides a viscosity of about 5 mPa·s or less;
about 0.5 mg/ml of edetate disodium; and
an acetate buffer, wherein the pharmaceutical composition has a pH of about 4.5.

53. The pharmaceutical composition of claim 1, comprising:
about 1 mg/ml or about 10 mg/ml of the peptide, or pharmaceutically acceptable derivative thereof, or pharmaceutically acceptable salt of any thereof, based on the amount of free peptide;
about 1 mg/ml or about 10 mg/ml of L-methionine, wherein the ratio of the concentration of methionine to the concentration of the peptide or pharmaceutically acceptable derivative thereof or pharmaceutically acceptable salt of any thereof, is about 1:1, based on the amount of free peptide;
sorbitol and sodium chloride as osmolarity regulating agents;
about 0.2 mg/ml of benzalkonium chloride;
hydroxypropyl methylcellulose as a thickening agent, optionally in an amount that provides a viscosity of about 5 mPa·s or less;
about 0.5 mg/ml of edetate disodium; and
an acetate buffer, wherein the pharmaceutical composition has a pH of about 4.5.

54. The pharmaceutical composition of claim 1, comprising:
about 1 mg/ml or about 10 mg/ml of the peptide, or pharmaceutically acceptable derivative thereof, or pharmaceutically acceptable salt of any thereof, based on the amount of free peptide;
about 1 mg/ml or about 10 mg/ml of L-methionine, wherein the ratio of the concentration of methionine to the concentration of the peptide or pharmaceutically acceptable derivative thereof or pharmaceutically acceptable salt of any thereof, is about 1:1, based on the amount of free peptide;
sorbitol as an osmolarity regulating agent;
about 0.2 mg/ml of benzalkonium chloride;
about 0.5 mg/ml of edetate disodium; and
an acetate buffer, wherein the pharmaceutical composition has a pH of about 4.5.

55. The pharmaceutical composition of claim 1, comprising:
about 1 mg/ml or about 10 mg/ml of the peptide, or pharmaceutically acceptable derivative thereof, or pharmaceutically acceptable salt of any thereof, based on the amount of free peptide;
about 1 mg/ml or about 10 mg/ml of L-methionine, wherein the ratio of the concentration of methionine to the concentration of the peptide or pharmaceutically acceptable derivative thereof or pharmaceutically acceptable salt of any thereof, is about 1:1, based on the amount of free peptide;
sorbitol as an osmolarity regulating agent;
about 0.2 mg/ml of benzalkonium chloride; and
about 0.5 mg/ml of edetate disodium.

56. The pharmaceutical composition of claim 1, comprising:
about 1 mg/ml or about 10 mg/ml of the peptide, or pharmaceutically acceptable derivative thereof, or pharmaceutically acceptable salt of any thereof, based on the amount of free peptide;
about 1 mg/ml or about 10 mg/ml of L-methionine, wherein the ratio of the concentration of methionine to the concentration of the peptide or pharmaceutically acceptable derivative thereof or pharmaceutically acceptable salt of any thereof, is about 1:1, based on the amount of free peptide;
sodium chloride as an osmolarity regulating agent;
about 0.2 mg/ml of benzalkonium chloride;
hydroxypropyl methylcellulose as a thickening agent, optionally in an amount that provides a viscosity of about 5 mPa·s or less; and
about 0.5 mg/ml of edetate disodium.

57. The pharmaceutical composition of claim 1, comprising:
about 1 mg/ml or about 10 mg/ml of the peptide, or pharmaceutically acceptable derivative thereof, or pharmaceutically acceptable salt of any thereof, based on the amount of free peptide;
about 1 mg/ml or about 10 mg/ml of L-methionine, wherein the ratio of the concentration of methionine to the concentration of the peptide or pharmaceutically acceptable derivative thereof or pharmaceutically acceptable salt of any thereof, is about 1:1, based on the amount of free peptide;
sorbitol as an osmolarity regulating agent;
about 0.2 mg/ml of benzalkonium chloride;
hydroxypropyl methylcellulose as a thickening agent, optionally in an amount that provides a viscosity of about 5 mPa·s or less; and
about 0.5 mg/ml of edetate disodium.

58. The pharmaceutical composition of claim 1, comprising:
about 1 mg/ml or about 10 mg/ml of the peptide, or pharmaceutically acceptable derivative thereof, or pharmaceutically acceptable salt of any thereof, based on the amount of free peptide;
about 1 mg/ml or about 10 mg/ml of L-methionine, wherein the ratio of the concentration of methionine to the concentration of the peptide or pharmaceutically acceptable derivative thereof or pharmaceutically acceptable salt of any thereof, is about 1:1, based on the amount of free peptide;
sorbitol and sodium chloride as osmolarity regulating agents;
about 0.2 mg/ml of benzalkonium chloride;
hydroxypropyl methylcellulose as a thickening agent, optionally in an amount that provides a viscosity of about 5 mPa·s or less; and
about 0.5 mg/ml of edetate disodium.

59. The pharmaceutical composition of claim 1, comprising:
about 1 mg/ml or about 10 mg/ml of the peptide, or pharmaceutically acceptable derivative thereof, or pharmaceutically acceptable salt of any thereof, based on the amount of free peptide;
about 1 mg/ml or about 10 mg/ml of L-methionine, wherein the ratio of the concentration of methionine to the concentration of the peptide or pharmaceutically acceptable derivative thereof or pharmaceutically acceptable salt of any thereof, is about 1:1, based on the amount of free peptide;
sodium chloride as an osmolarity regulating agent;
about 0.2 mg/ml of benzalkonium chloride; and
hydroxypropyl methylcellulose as a thickening agent, optionally in an amount that provides a viscosity of about 5 mPa·s or less.

60. The pharmaceutical composition of claim 1, comprising:
about 1 mg/ml or about 10 mg/ml of the peptide, or pharmaceutically acceptable derivative thereof, or pharmaceutically acceptable salt of any thereof, based on the amount of free peptide;
about 1 mg/ml or about 10 mg/ml of L-methionine, wherein the ratio of the concentration of methionine to the concentration of the peptide or pharmaceutically acceptable derivative thereof or pharmaceutically acceptable salt of any thereof, is about 1:1, based on the amount of free peptide;
sorbitol as an osmolarity regulating agent;
about 0.2 mg/ml of benzalkonium chloride; and
hydroxypropyl methylcellulose as a thickening agent, optionally in an amount that provides a viscosity of about 5 mPa·s or less.

61. The pharmaceutical composition of claim 1, comprising:
about 1 mg/ml or about 10 mg/ml of the peptide, or pharmaceutically acceptable derivative thereof, or pharmaceutically acceptable salt of any thereof, based on the amount of free peptide;
about 1 mg/ml or about 10 mg/ml of L-methionine, wherein the ratio of the concentration of methionine to the concentration of the peptide or pharmaceutically acceptable derivative thereof or pharmaceutically acceptable salt of any thereof, is about 1:1, based on the amount of free peptide;
sorbitol and sodium chloride as osmolarity regulating agents;
about 0.2 mg/ml of benzalkonium chloride; and
hydroxypropyl methylcellulose as a thickening agent, optionally in an amount that provides a viscosity of about 5 mPa·s or less.

62. The pharmaceutical composition of any one of claims 1-61, wherein the composition comprises an acetate salt of the peptide.

63. The pharmaceutical composition of any one claims 1-61, wherein the composition comprises a pharmaceutically acceptable derivative of the peptide having an N-terminal acetyl moiety.

64. The pharmaceutical composition of any one of claims 1-61 or 63, wherein the composition comprises a pharmaceutically acceptable derivative of the peptide having a C-terminal amidate moiety or a C-terminal adamantane moiety.

65. The pharmaceutical composition of any one claim 1-61, wherein the composition comprises a pharmaceutically acceptable derivative of the peptide selected from:
N-acetyl-Met-Glu-His-Phe-Pro-Gly-Pro;
N-acetyl-Met-Glu-His-Phe-Pro-Gly-Pro-amidate, and
N-acetyl-Met-Glu-His-Phe-Pro-Gly-Pro-adamantane-CONH₂.

66. The pharmaceutical composition of any one of the preceding claims, wherein the composition does not include any parabens or salts thereof.

67. The pharmaceutical composition of any one of the preceding claims, provided in or packaged with a nasal spray drug delivery device.

68. The pharmaceutical composition of claim 67, wherein the nasal spray drug delivery device is configured to deliver a target dose volume of about 100 μL.

69. A method of treatment, comprising administering a pharmaceutical composition according to any one of claims 1-68 to a subject in need thereof by intranasal administration.

70. The method of claim 69, wherein the pharmaceutical composition is administered from a nasal spray drug delivery device.

71. The method of any one of claims 69-70, wherein the pharmaceutical composition is administered at a target dose volume of about 100 μL.

72. The method of any one of claims 69-71, wherein the method is for treating one or more conditions selected from intellectual-mental disorders in vascular malformations of the brain, dyscirculatory encephalopathy, transient ischemic attack (TIA), neurotic disorders, glaucoma, optic nerve disorders, minimal brain dysfunctions in children, attention deficit hyperactivity disorder (ADHD), mental fatigue, multiple sclerosis, perinatal damage of CNS with atonic-astatic syndrome, and organic mental disorders in children, or for promoting recovery after craniocerebral injury or for promoting recovery after narcosis, or for stroke management or treatment after neurosurgery.

73. An aqueous pharmaceutical composition according to any one of claims 1-68, for use in treating one or more conditions selected from intellectual-mental disorders in vascular malformations of the brain, dyscirculatory encephalopathy, transient ischemic attack (TIA), neurotic disorders, glaucoma, optic nerve disorders, minimal brain dysfunctions in children, attention deficit hyperactivity disorder (ADHD), mental fatigue, multiple sclerosis, perinatal damage of CNS with atonic-astatic syndrome, and organic mental disorders in children, or for promoting recovery after craniocerebral injury or for promoting recovery after narcosis, or for stroke management or treatment after neurosurgery.

74. Use of peptide Met-Glu-His-Phe-Pro-Gly-Pro (SEQ ID NO: 1), or a pharmaceutically acceptable derivative thereof, or a pharmaceutically acceptable salt of any thereof, in the preparation of a medicament for treating one or more conditions selected from intellectual-mental disorders in vascular malformations of the brain, dyscirculatory encephalopathy, transient ischemic attack (TIA), neurotic disorders, glaucoma, optic nerve disorders, minimal brain dysfunctions in children, attention deficit hyperactivity disorder (ADHD), mental fatigue, multiple sclerosis, perinatal damage of CNS with atonic-astatic syndrome, and organic mental disorders in children, or for promoting recovery after craniocerebral injury or for promoting recovery after narcosis, or for stroke management or treatment after neurosurgery, wherein the medicament comprises an aqueous pharmaceutical composition according to any one of claims 1-68.
